(19) United States
(12) Patent Application Publication
(10) Pub. No.: US 2008/0214551 A1
(43) Pub. Date: Sep. 4, 2008

(54) Treatment of Burns

(75) Inventors: Russell Taylor, Berkshire (GB); Paul Crees, Berkshire (GB)

Correspondence Address: MYERS BIGEL SIBLEY & SAJOVEC, PO BOX 37428, RALEIGH, NC, 27627, US

(73) Assignee: AQ+ PLC, Glasgow (GB)

(21) Appl. No.: 11/632,682

(22) PCT Filed: Jul. 19, 2005
(86) PCT No.: PCT/GB05/02849

§ 371 Date: Nov. 6, 2007

(30) Foreign Application Priority Data
Jul. 19, 2004 (GB) 0415985.1

(51) Int. Cl. A61K 31/47; A61K 31/4412; A61K 31/513; A61K 31/52; A61K 31/498; A61K 31/519

(52) U.S. Cl. 514/250; 514/348; 514/269; 514/249; 514/311; 514/263.1

(57) ABSTRACT

The present invention provides methods of treatment of burns, which comprise the step of applying to the burns of a subject a therapeutically effective amount of a metal ion chelating agent.

This invention relates to manufacture and applications of metal ion chelating compositions for the treatment of various burns conditions in human and non-human animals.

Burns are a major form of injury in today's society. They can come from many sources including inter alia: excessive exposure to radiation, e.g. solar radiation resulting in sunburn, thermal radiation, welding flash, fires, electrical discharge, contact with chemicals, friction, contact with very hot objects such as cooking apparatus elements or hot fluids such as scalding water, hot oil, etc. Problems often arise due to inappropriate treatment, the associated pain, and formation of scar tissue. The greatest numbers of people who suffer the most from burns are children who are attempting to handle a situation where either hot water or oil is involved.

Burns are generally classified accordingly to their seriousness and extent.

First Degree Burns are the mildest and normally only affect the epidermis. The burn site is red, painful, dry, no blisters, very sensitive to touch and the damaged skin may be slightly moist from the leakage of fluid in the deeper layers of the skin. At this point the sensory nerve ends are also exposed and creating pain. Mild sunburn is typical of a First degree Burn situation,

Second Degree Burns is where both the Epidermis and Dermis are affected. The damage is deeper and blisters usually appear on the skin. The skin is still painful and sensitive, as the nerves have been affected as well as the sebaceous glands in the area.

Third degree burns are the most serious, as the tissues in all layers of the skin are dead. Normally the damaged area goes down into the subcutaneous tissue. Usually there are no blisters but the burnt surface can have several types of appearance, from white to black (charred) or bright red from blood in the bottom of the wound. In most cases it can penetrate down through the superficial fascia, and into the muscle layers where various arteries and veins may be affected. Because the skin nerves are damaged the burn can be quite painless and on touching the skin sometimes it has no sensation what so ever. The lack of sensation or blanching of the skin blood vessels on pressure indicates damaged skin.

In all cases bacterial infection is a significant risk and the bacteria Pseudomonas aeroginosa is generally the most prevalent bacteria that can inhabit burn areas. This bacteria is a very hardy type and is very difficult to dislodge from the burn area with the result sometimes that healing of the area through various procedures including skin grafts is not possible, as the underlying basal tissue has been contaminated and damaged and substantial scar tissue can result from this bacterial infection.

The normal treatment for burns is initially to run cold water over the damage area with the intention of lowering the temperature. Normally with first-degree burns no further treatment is required and the skin repairs itself naturally within a few days.

With second degree burns after running cold water over the burn area, conventionally an antibacterial ointment or salve is applied to assist in the healing process and normally the damage area will resolve itself back to normal within 3 weeks. Sometimes if blisters are present and they burst they may need to be trimmed to get rid of the dead skin.

With third degree burns depending on the severity will dictate the treatment. Normally extensive medication is required under hospital conditions to ensure that the damaged area is treated properly to try and save as much as possible. Skin grafts in this case will probably have to be done after a period of stabilisation. These burns normally become infected quickly if not treated and can result in gangrene, loss of a limb, or sepsis.

With third degree burns, rehabilitation of the damaged area normally leaves scar tissue as the skin cover, which is taut and sometimes results in restriction of movement.

It is an object of the present invention to avoid or minimize one or more of the above problems.

There has previously been proposed in our earlier Patent publication WO 03/032944, various topical chelating compositions suitable for use in combating antibiotic-resistant infections and contamination of the skin and open wounds. There has been no previous suggestion, however, of the possible utility of such compositions for other conditions.

We have now found that compositions based on the use of chelating compounds as disclosed in WO 03/032944 (the contents of which are hereby incorporated herein by reference thereto), have significant beneficial properties in treating various burn conditions, and in particular in reducing the risk of infection which is a particular problem in the management and treatment of burn conditions, as well as the reduction or elimination of pain associated therewith, and the unique action in the promotion of natural skin and tissue regrowth, and vascularization thereof.

We have found that the chelating compound will coat any bacteria present and deprive it of metal ions, which the bacteria rely on for food. This means that any infection that may be present from Pseudomonas aeroginosa or other bacteria, is treated immediately and quickly brought under control.

Thus in a first aspect the present invention provides the use of a metal ion chelating agent for the manufacture of a medicament for the treatment of burns.

For the avoidance of doubt, references to “burns” or “burns conditions” herein are intended to encompass the full range of such conditions, including those resulting from: excessive exposure to radiation, e.g. solar radiation resulting in sunburn, thermal radiation, welding flash, fires, electrical discharge, contact with chemicals, friction, contact with very hot objects such as cooking apparatus elements or hot fluids such as scalding water, hot oil, etc, unless the contrary is indicated.

Preferably, the metal ion chelating agent is used at a pH substantially higher than the pH of the blood in the patient. In general the pH of blood in a normal patient is around 7.4. When the pH of the treatment composition is higher than that of the blood, it draws blood into the burn area to which the composition has been applied, thereby promoting healing of the burn and skin and tissue regrowth. In general the greater the pH differential between the blood pH, and the composition pH, the stronger the effect on drawing in blood, and the greater the healing promotion effect. On the other hand, excessively high pH values, will give rise to toxicity and/or other injurious effects on the body. In general we have found that the pH should be in the range from 8.0 to 9.6, preferably from 9.0 to 9.5, most preferably from 9.2 to 9.4.

In a further aspect the present invention provides a method for the treatment of burns, comprising the application to the burns on a human or animal suffering from burns, of an effective dose of a metal ion chelating agent.

Preferred chelating agents can chelate various different metal ions and thereby attack bacteria by multiple, direct and indirect, routes, thereby maximizing protection of the burns area against infection during the recovery period. More particularly, it is preferable for the chelating agent(s) used to form a chelate with a plurality of metal ions selected from Mg²⁺, Fe²⁺, Cu²⁺, Zn²⁺, Mn²⁺, Ni²⁺, and Se²⁺. 8-hydroxyquinoline has been found to have a particularly broad spectrum of activity, chelating most metals apart from sodium, potassium and calcium.

Preferably the metal ion chelating agent is a heteropolar compound comprising at least one unsaturated heterocyclic six-membered ring in which at least one heteroatom moiety acts as a hydrogen acceptor and in which said compound also comprises at least one hydrogen donor moiety, conveniently a hydroxyl group, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound.

In general the preferred metal ion chelating agent is a hetero aryl compound having at least one nitrogen in the ring structure and at least one hydroxyl substituent disposed on the ring structure so as to provide together, a chelating function. Preferred metal ion chelating agents are selected from optionally substituted 2,3-dihydroxypyridine; 4,6-dihydroxypryrimidine; 2-pteridinol; 2, 4-quinolindiol; 2, 3-dihydroxyquinoxalin; 2,4-pteridinediol; 6-purinol; 3-phenanthridinol; 2-phenanthrolinol; 2-phenazinilol, and most preferred is 8-hydroxyquinoline. 8-hydroxyquinoline has the advantage of forming metal ion chelates with a particularly broad range of different metal ions.

In a further aspect the present invention provides a pharmaceutical composition for topical application comprising said metal ion chelating agent in a pharmaceutically acceptable carrier therefor, preferably an aqueous based carrier. Suitable aqueous based carriers will generally also include an intermediate diluent, wetting agent, a thickener where needed, and a pH controller, for providing a composition pH which is higher than blood pH, as discussed hereinbefore. The compositions will generally be in the form of liquids, gels or pastes and will generally comprise from 0.0031% to 0.20% w/w, preferably from 0.02 to 0.1% (???) w/w, of the chelating agent with higher concentrations tending to give a faster and/or more effective healing.

At the same time the composition coats the nerve receptors to neutralise the pain. This means that the suffering due to the pain from the damaged area is minimised.

For use on first-degree burns, there is generally employed a liquid form composition, which conveniently is sprayed onto the damaged area and/or may be wiped across the damaged area to help ensure complete coverage. This will typically relieve the pain within a few minutes and generally within a period of as little as 24 hours, there will be no visible sign of any burn area or redness.

For the treatment of second or third degree burn areas, the use of a gel composition, which is of a pourable viscosity, or a paste composition, is generally preferred. It is important to ensure that the area where the gel or paste is applied remains moist, as that will help ensure that the nerve receptors remain covered with the composition, and pain relief is substantially maintained.

With second-degree burns, after the application of the composition on the area and even unburst blisters, it will generally be found that within 24 hours, the blisters have reduced in size, the redness substantially disappeared, and the pain has substantially disappeared. If the blisters have burst then the skin is repaired within 2-3 days with repeated applications of the composition.

With third degree burns the composition will help ensure that the burns area is protected from infection, and healing proceeds. The tissue and skin will start to rebuild, and after a few days the skin and tissue take on a new colour appearance as healing progresses. The period of time required to complete the healing process will generally depend on the severity of the burn injury at the damaged area.

It will be appreciated that the choice of other components of the composition may be limited by the nature of the metal ion chelating agent. Thus, for example, since the preferred metal ion chelating agent 8-hydroxyquinoline is generally insoluble or only poorly soluble in aqueous solution. Suitable aqueous based compositions of 8-hydroxyquinoline can be prepared by using an intermediate solvent such as a polyol, including glycols, preferably propylene glycol, glycerine, or sorbitol, and a wetting agent. Those skilled in the art will appreciate that a wide range of wetting agents are available that may be used which would give solubility of the metal ion chelating agent in glycol, including inter alia Polyoxyethylene Sorbitan Fatty Acid Ester T20, T40, T60 and T80 (Polysorbate), and C9-C11 Alcohol ethoxylate (Symperonic 91/8, or more preferably, Symperonic 91/6).

It will be appreciated that a range of different proportions of the various components of the aqueous based compositions may be used depending on the solubilities of the metal ion chelating agents used, the final concentration required etc. In general we have found that the amount of wetting agent used is relatively sensitive. In the case of the intermediate solvent (glycol etc), once a required minimum amount sufficient for solubilisation of the metal ion chelating agent in the water is present, then the amount of this intermediate solvent can be readily increased further, though there is normally no particular advantage in doing so.

Advantageously there is also included a pH controller, in order to ensure an alkaline pH in the composition, which is higher than blood pH, as discussed hereinbefore, most preferably a pH in the region of 9.2 to 9.4. Any convenient physiologically acceptable pH control material may be used. The pH controller may simply be an alkali such as KOH or NaOH. Preferably, though, there is used EDTA, conveniently in the form of the DiSodium or TetraSodium salt (DSEDTA or TSEDTA, respectively).

In the case of 8-hydroxyquinoline we have found that suitable proportions which may be used in a liquid, aqueous-based, composition of the invention suitable for use in the treatment of burns, would in general have the following composition:

| Component | Weight |
|---|---|
| Primary chelating agent | 1 part |
| (8-hydroxyquinoline) | |
| Secondary chelating agent | 0.1 part |
| (EDTA) | |
| Wetting agent | 4 +/− 5% parts |
| Diluent | at least 20 preferably 40 |
| Deionised Water | as required to obtain the |
| | final concentration |
| | required. |
| pH controller (DSEDTA or TSEDTA) | as required to achieve a |
| | pH of 9.2 to 9.4. |
| Thickener | as required for the |
| | desired consistency |

Further preferred features and advantages of the present invention will appear from the following detailed examples given by way of illustration of some preferred embodiments.

### EXAMPLE 1

### Method of Preparation of Concentrate

10 gm of 8-hydroxyquinoline (chelating compound) and 0.5 gram of Ethylene Diamine Tetra Acetic Acid (pH controller), were dissolved at 70 degrees centigrade in 50 grammes of a wetting agent selected from: Polyoxyethylene Sorbitan Fatty Acid Ester T20, T40, T60 and T80 (Polysorbate), and C9-C11 Alcohol ethoxylate (Symperonic 91/6), with 200 grams of a water soluble non-aqueous diluent selected from Propylene Glycol, Glycerine and Sorbitol. Once solution has been achieved, a further quantity of the glycol or glycerine or Sorbitol diluent was added to make up to a solution of 500 grammes and then cooled giving 500 g of a concentrate containing a mixture of 2.10% primary and secondary chelating compounds.

### EXAMPLE 2

### Preparation of Liquid Spray Composition

Take one part of the 2.1% chelating concentrate from Example 1 and dilute in 39 parts of deionised water, then the pH of this composition is adjusted to pH 9.2/9.4 by the addition of Tetra Sodium Ethylene Diamine Acetic Acid. The strength of this preparation is 525 ppm of chelating compound.

This product is suitable for treating first degree burns such as sunburn, which is a common complaint during summer, or for welding flash that occurs with arc welders in engineering companies. The composition is sprayed onto the area of the burn as quickly as possible after the burn has occurred, and thereafter is desirably reapplied at intervals of 10 to 15 minutes until the skin can be touched with no pain.

### EXAMPLE 3

### Preparation of a Gel Composition

Take one part of the 2.1% chelating concentrate from Example 1 and dilute in 39 parts of deionised water. A hydroxypropylcellulose thickener, is then added at the rate of 1% w/v, to produce a pourable gel composition and then the pH is adjusted in the composition to 9.2/9.4 with Tetra Sodium Ethylene Diamine Tetra Acetic Acid (TSEDTA). The strength of this preparation is 519 ppm of chelating compound.

The application of this gel is dependent on the burn and the person concerned but a coating of the gel should be at least 2-3 mm thick on each application. It is necessary to keep the gel on the burn area moist to prevent pain coming back so if necessary a suitable dressing or covering (conveniently in the form of disposable diaper material), is placed over the area with a plastics film wrap around it, to prevent the gel from drying out.

### EXAMPLE 4

### Preparation of a Paste Composition

Take one part of the 2.1% chelating concentrate from Example 1 and dilute in 39 parts of deionised water. A hydroxypropylcellulose thickener, is then added at the rate of 2% w/v, to produce a viscous paste and then the pH is adjusted in the composition to 9.2/9.4 with Tetra Sodium Ethylene Diamine Tetra Acetic Acid (TSEDTA). The strength of this preparation is 515 ppm of chelating compound.

The application of this paste is dependent on the burn area and the person concerned but a coating of the paste should generally be at least 2-3 mm thick on each application. It is necessary to keep the paste on the burn area moist so if necessary a suitable dressing or covering (conveniently in the form of disposable diaper material), is placed over the burn area with a plastics film wrap around it, to prevent the paste from drying out.

### EXAMPLE 5

### Treatment of Third Degree Burns

A 48 year old male subject with a third degree burns area of around 155 cm², on his arm, resulting from a hot oil spillage, had a paste composition according to Example 4, applied to the burns area, twice a day. Bacterial infection was overcome after the first day, and visible skin regrowth was present by the third day. By the 6^{th} day of treatment, natural skin regrowth was present over almost the whole of the burns area.

### EXAMPLE 6

### Treatment of Third Degree Burns

An 11 year old female subject with 70% third degree electrical burns, resulting from contact with a broken HT Voltage transmission cable, had been receiving conventional treatment for a period of about 8 months and had received extensive skin grafts. Certain areas, though, had become infected with, and were resisting conventional treatment. These areas had a paste composition according to Example 4, applied thereto, twice a day. By the 8^{th} day of treatment, the bacterial infection had been brought under control, and natural skin regrowth was present over almost the whole of the treated area.

### EXAMPLE 7

### Treatment of First Degree Burns

A senior male subject with first degree burns across two fingers resulting from scalding with boiling water, had the affected area sprayed 3 times at intervals of 15 minutes with a liquid composition according to Example 2, after first running cold water over the fingers for several minutes. After this the pain substantially disappeared, and by the following day there was only a slight tenderness to touch, and a slight reddening of the burnt area, with no blister formation.

1. A method of treatment of burns, said method comprising the step) of applying to the burns of a subject, a therapeutically effective amount of a metal ion chelating agent.

2. The method according to claim 1 wherein said metal ion chelating agent is used together with a physiologically acceptable pH control agent providing an alkaline pH higher than 7.4.

3. The method according to claim 2, wherein said alkaline pH is in the range from 8.0 to 9.6.

4. The method according claim 1 wherein said metal ion chelating agent is a heteropolar compound comprising at least one unsaturated heterocyclic six-membered ring in which at least one heteroatom moiety acts as a hydrogen acceptor and wherein said heteropolar compound also comprises at least one hydrogen donor moiety, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound.

5. The method according to claim 1 wherein said metal ion chelating agent is a hetero aryl compound having at least one nitrogen in the ring structure and at least one hydroxyl substituent disposed on the ring structure so as to provide together, a chelating function.

6. The method according to claim 5 wherein said metal ion chelating agent is selected from optionally substituted 2,3-dihydroxypyridine; 4,6-dihydroxypryrimidine; 2-pteridinol; 2,4-quinolindiol; 2,3-dihydroxyquinoxalin; 2,4-pteridinediol; 6-purinol; 3-phenanthridinol; 2-phenanthrolinol; 2-phenazinilol, and 8-hydroxyquinoline.

7. The method according to claim 6 wherein said metal ion chelating agent is 8-hydroxyquinoline.

8. The method according to claim 1 wherein the metal chelating agent is in a pharmaceutical formulation further comprising a wetting agent.

9. The method according to claim 8 wherein the wetting agent is selected from Polyoxyethylene Sorbitan Fatty Acid Ester T20, T40, T60 and T80 (Polysorbate), and C9-C11 Alcohol ethoxylate (including Symperonic 91/8, and Symperonic 91/6).

10. The method according to claim 1 wherein the metal chelating agent is in a pharmaceutical formulation further comprising an intermediate solvent in the form of a non-aqueous water soluble solvent.

11. The method according to claim 10 wherein said intermediate solvent is a polyol.

12. The method according to claim 11 wherein said intermediate solvent is selected from monoethylene glycol, propylene glycol glycerine, and sorbitol.

13. The method according to claim 1 wherein the metal chelating agent is in a pharmaceutical formulation further comprising a thickener in said medicament.

14. The method according to claim 13 wherein said thickener is a hydroxypropylcellulose thickener.

15. The method according to claim 13 wherein said thickener is a dehydroxanthan gum thickener.

16. The method according to claim 1 wherein the metal chelating agent is in a pharmaceutical formulation comprising 1 part by weight of 8-hydroxyquinoline, 4+5% parts by weight of wetting agent, at least 20 parts by weight of glycol, and water.

17. The method according to claim 1 wherein the metal chelating agent is in a pharmaceutical formulation in the form of a liquid, spray, cream, gel or paste.

18. The method according to claim 1 in wherein the metal ion chelating agent is in a pharmaceutical formulation at a concentration of from 0.0031% to 0.20% w/w of the chelating agent(s).

19. The method according to claim 18 in which said metal ion chelating agent is at a concentration of from 0.02 to 0.1% w/w of the chelating agent.

20. The method according to claim 2, wherein the alkaline PH is in the range from 9.0 to 9.5.

21. The method according to claim 20 wherein the alkaline PH is in the range from 9.2 to 9.4.

22.-23. (canceled)
